(19) United States
(12) Patent Application Publication
(10) Pub. No.: US 2024/0350560 A1
(43) Pub. Date: Oct. 24, 2024

(54) COMPOSITION COMPRISING PASTEURIZED AKKERMANSIA MUCINIPHILA FOR THE TREATMENT OR PREVENTION OF GUT CONTRACTILITY DISORDERS, IN PARTICULAR DUODENAL CONTRACTION AMPLITUDE DISORDER

(71) Applicant: THE AKKERMANSIA COMPANY, Mont-Saint-Guibert (BE)

(72) Inventors: Patrice CANI, Mont-Saint-Guibert (BE); Amandine BROCHOT, Mont-Saint-Guibert (BE)

(21) Appl. No.: 18/684,681

(22) PCT Filed: Aug. 2, 2022
(86) PCT No.: PCT/EP2022/071707

§ 371 Date: Feb. 18, 2024

(30) Foreign Application Priority Data
Aug. 19, 2021 (BE) BE2021/5663

(51) Int. Cl. A61K 35/741; A23L 33/105; A23L 33/135; A61K 36/258; A61K 36/45; A61K 36/47; A61K 36/61; A61K 36/63; A61K 36/73; A61K 36/752; A61K 36/82; A61P 1/00

(52) U.S. Cl. CPC A61K 35/741; A23L 33/105; A23L 33/135; A61K 36/258; A61K 36/45; A61K 36/47; A61K 36/61; A61K 36/63; A61K 36/73; A61K 36/752; A61K 36/82; A61P 1/00

(57) ABSTRACT

Disclosed are compositions comprising pasteurized Akkermansia for the treatment or prevention of gut contractility disorders, in particular for reducing the duodenal amplitude of contractions.

### FIELD OF THE INVENTION

The present invention relates to a composition comprising pasteurized Akkermansia muciniphila for the treatment or prevention of contractility disorders, in particular duodenal contraction amplitude.

### BACKGROUND

Disorder of the gut contraction are on the rise. For example, diabetes is generally associated with an intestinal hyper-contractility. This may favor hyperglycemia and insulin-resistance.

### Source:

Knauf, C., Abot, A., Wemelle, E. & Cani, P. D. Targeting the Enteric Nervous System to Treat Metabolic Disorder? ‘Enterosynes’ as Therapeutic Gut Factors. Neuroendocrinology (2019) doi:10.1159/000500602.

Fournel, A. et al. Apelin targets gut contraction to control glucose metabolism via the brain. Gut 66, 258-269 (2017).

Abot, A. et al. Galanin enhances systemic glucose metabolism through enteric Nitric Oxide Synthase-expressed neurons. Mol Metab 10, 100-108 (2018).

On the other hand, pasteurized Akkermansia has been described on having a positive effect on the gut-barrier function.

WO2017042347A1 to UCL and University of Wageningen discloses the use of pasteurized Akkermansia for treating obesity and diabetes. However, the use for treating gut contractility disorders is not disclosed.

WO2017178496A1 to University of Wageningen discloses Akkermansia glycaniphilus for use in preventing and/or treating a disorder selected from the group consisting of amongst others, diabetes, obesity, or irritable bowel syndrome (IBS), and other diseases related to compromised barrier function.

However, the use of a composition comprising pasteurized Akkermansia for treating or preventing gut contractility disorders, in particular duodenal contraction amplitude disorder is not disclosed.

Hence, most of the Akkermansia-related patent publication only address the composition of the microbiome in the gut and the gut barrier-function, but not the biomechanics of the gut and the gut contractions and its stretching and motility.

Therefore, there is still an urgent need to provide compositions for treating gut contractility disorders, in particular, for reducing the amplitudes of duodenal gut contraction.

### SHORT DESCRIPTION OF THE INVENTION

The present inventors have surprisingly found that a composition comprising pasteurized Akkermansia may be effective in modulating the biomechanics, contraction and stretching of the gut and, thus in treating or preventing gut contractility disorders, in particular duodenal contraction amplitude disorder.

In fact, the oral administration of pasteurized Akkermansia muciniphila substantially has reduced the duodenal contraction amplitude while leaving the duodenal contraction frequency substantially unchanged. Moreover, the oral administration of pasteurized Akkermansia muciniphila substantially has reduced glucose absorption in the jejunum.

Accordingly, a first aspect of the invention is a composition, comprising pasteurized Akkermansia for use in the prevention or treatment of gut contractility disorders.

Another aspect is the composition for use in the prevention or treatment of gut contractility disorders, wherein the pasteurized Akkermansia is Akkermansia muciniphila.

Un autre aspect est la composition destinée à être utilisée dans le traitement ou dans la prévention des troubles de la contractilité intestinale, dans laquelle les Akkermansia, en particulier les Akkermansia muciniphila ne sont pas pasteurisés.

Another aspect is the composition for use in the prevention or treatment of gut contractility disorders, wherein the Akkermansia, in particular the Akkermansia muciniphila are not pasteurized.

Another aspect is a composition for use in the prevention or treatment of gut contractility disorders, wherein the duodenal amplitude is controlled, modulated, or reduced in patients suffering from gut contractility disorders.

Another aspect is the composition for use in the prevention or treatment of gut contractility disorders, wherein the jejunum glucose absorption is controlled, modulated, or reduced in patients suffering from gut contractility disorders.

Another aspect is the composition for use in the prevention or treatment of gut contractility disorders, wherein the jejunum glucose absorption is controlled or reduced in diabetes or obesity patients suffering from gut contractility disorders.

Another aspect is the composition for use in the prevention or treatment of gut contractility disorders, wherein pasteurized Akkermansia is administered for 2 to 10 days, preferably for 3 to 7 days and even more preferably for 4 to 6 days.

In another aspect, the composition is administered for one week or longer, for two weeks or longer or even permanently.

Another aspect is the composition for use in the prevention or treatment of gut contractility disorders, wherein the pasteurized Akkermansia is administered in an amount from 1·10⁴ to 1·10¹² cells per day, more preferably from 1·10⁵ cells to 1·10¹¹ cells per day, and even more preferably from 1·10⁶ to 5·10¹⁰ cells per day.

Another aspect is the composition for use in the prevention or treatment of gut contractility disorders according to any of the preceding claims, wherein the pasteurized Akkermansia is administered in an amount from 1·10⁸ to 5·10¹⁰ cells per day.

Another aspect is the composition for use in the prevention or treatment of gut contractility disorders, further comprising one or more ingredients chosen from group consisting of probiotic, bacteria, yeast, microorganisms, prebiotic, or a combination thereof.

Another aspect is the composition for use in the prevention or treatment of gut contractility disorders, wherein the composition further comprising a mineral or a vitamin or a combination thereof.

Another aspect is the composition for use in the prevention or treatment of gut contractility disorders, wherein the composition further comprises a pharmaceutically acceptable carrier or a food grade carrier.

Another aspect is the composition for use in the prevention or treatment of gut contractility disorders according to any of the preceding claims, wherein the composition is orally administered.

Another aspect is the composition for use in the prevention or treatment of gut contractility disorders, wherein the composition is a cosmetic composition, a nutritional composition, a food product, a dietary complement, a medical food, or a medicament.

Another aspect is the composition for use in the prevention or treatment of gut contractility, wherein the composition further comprises a plant extract, chosen from the group consisting of Camellia sinensis, Aronia melanocarpa, Emblica officinalis, Olea Europa, Citrus bergamia, Vaccinium macrocarpon, Myrciaria dubia, red Panax ginseng, Vaccinium oxycoccos, Vaccinium macrocarpon.

Another aspect is the non-medical use of the composition comprising pasteurized Akkermansia for controlling, modulating, or reducing gut contractility disorders.

### Definitions

“Treatment” means reducing, controlling, modulating, or alleviating at least one adverse effect or symptom of a disease, disorder or condition. This term thus refers to both therapeutic treatment and prophylactic or preventative measures.

“Prevention” means preventing in the sense of keeping from happening or reducing the risk of a disease or condition.

“Effective amount” or “therapeutically effective amount” refers to level or amount of agent that is aimed at, without causing significant negative or adverse side effects to the target, delaying or preventing the onset of a metabolic disorder, slowing down or stopping the progression, aggravation, or deterioration of one or more symptoms of the metabolic disorder; bringing about ameliorations of the symptoms of the metabolic disorder; reducing the severity or incidence of the metabolic disorder; curing the metabolic disorder; or restoring the normal amount and/or proportion of Akkermansia muciniphila in the gut of the subject to be treated.

“Akkermansia muciniphila” refers to the mucin-degrading bacteria identified by Derrien (Derrien et al., 2004. Int. J. Syst. Evol. Microbiol. 54:1469-1476). Cells are oval-shaped, non-motile and stain Gram-negative. Akkermansia muciniphila may also be referred as Akkermansia spp. or Akkermansia-like bacteria. It belongs to the Verrucomicrobia phylum. It is generally accepted that strains with a nucleotide similarity as experimentally determined by DNA-DNA hybridization of about 70% can be considered as the same species—this corresponds to an average nucleotide identity (ANI) of approximately 95%.

“Pasteurized Akkermansia muciniphila” refers to Akkermansia muciniphila submitted to a heating treatment. In one embodiment, pasteurized Akkermansia muciniphila refers to Akkermansia muciniphila which was heated at a temperature from 50° C. to 100° C. for at least 10 minutes.

“TFU” or “Total Fluorescent Units” means the number of cells as determined by their fluorescence radiance after staining.

In a preferred embodiment, the TFU is measured by flow cytometry. For example, in a first step, aliquots of biomass batches are rehydrated in PBS and stained with Syto 9 and propidium iodide according manufacturer protocol (LIVE/DEAD® BacLight™ Bacterial Viability Kit, Thermofisher). The TFU may then be obtained by analyzing the stained samples on Attune NxT flow cytometer.

“Probiotics” refers to live microorganisms which, when administered in an effective amount, provide a beneficial effect on the health or well-being of a subject. In one embodiment, these health benefits are associated with improving the balance of human or animal microbiota in the gastrointestinal tract, or restoring normal microbiota.

“Prebiotic” refers to a substance, such as, for example, a substance which may not be digested by humans, but which modulates composition and/or activity of the gut microbiota through its metabolization by microorganisms in the gut, thus conferring a beneficial physiological effect on the host.

“Subject” refers to an animal, preferably a mammal, more preferably a human or an animal.

“Gut contractility disorders” refers to a subject situation wherein the functioning of the gut is impaired as compared to a healthy individual. Gut contractility disorders may include a modulation of the amplitudes or the frequency of the gut contraction and stretching. In a preferred embodiment, gut contractility concerns the amplitudes of contractions of the duodenum.

### DETAILED DESCRIPTION OF THE INVENTION

The applicant herein shows the beneficial effects on gut contractility disorders after administration of a composition comprising pasteurized Akkermansia muciniphila.

In one embodiment, pasteurized Akkermansia muciniphila of the invention are non-viable cells. As used herein, “non-viable cells” means cells that are not able to proliferate. Examples to visualize or count cells of Akkermansia muciniphila have been provided by Derrien et al. (2008. Appl. Environ. Microbiol. 74:1646-8), Derrien et al. (2011. Frontiers Microbiol. 2:166-175) or Reunanen et al. (2015. Appl. Environ. Microbiol. 81(11):3655-62).

The composition of the invention may also comprise a pharmaceutically acceptable excipient or a food grade carrier, for example solvents, dispersion media, coatings, isotonic and absorption delaying agents and the like. For human administration, preparations should meet general safety and purity standards as required by FDA Division of Biological Standards.

The present invention also relates to a medicament, medical food, food, or dietary compliment comprising an effective amount of pasteurized Akkermansia muciniphila.

Another object of the invention is a method for restoring a normal proportion of Akkermansia muciniphila, in the gut of a subject in need thereof, wherein said method comprises administering an effective amount of Akkermansia muciniphila to the subject.

In one embodiment of the invention, the composition of the invention is administered at least once a week, preferably at least twice a week, more preferably at least three times a week, and even more preferably at least four times a week. In another embodiment, the composition of the invention is administered at least once a day, and preferably at least twice a day.

In one embodiment, the composition of the invention is administered for 1 week, preferably during 2, 3, 4, 5, 6, 7 or 8 weeks or more or even permanently.

In one embodiment of the invention, the daily dosage of Akkermansia muciniphila administered is from 1·10² to about 1·10¹⁵ TFU/day, preferably from about 1·10⁴ to about 1·10¹² TFU/day, more preferably from about 1·10⁵ to about 1·10¹¹ TFU/day and even more preferably from about 1·10⁶ to about 1·10¹⁰ TFU/day.

In another embodiment of the invention, the daily dosage of pasteurized Akkermansia muciniphila is from 1·10⁶ to about 1·10¹² cells/day, preferably from about 1·10⁸ to about 1·10¹⁰ cells/day, more preferably from about 1·10⁹ to about 5·10¹⁰ cells/day.

The present invention also relates to the cosmetic use of pasteurized Akkermansia muciniphila for reduction of gut contractility disorders.

Another object of the invention is thus a non-medical use of a composition comprising an effective amount of pasteurized Akkermansia muciniphila and the use thereof for reduction of gut contractility disorders.

Another object of the invention is a method for controlling, modulating, treating, or reducing gut contractility disorders, wherein the method comprises administering pasteurized Akkermansia to a subject in need thereof, preferably in a therapeutically efficient dose without causing treatment limiting side effects.

Another object of the invention is a method for controlling, modulating, treating, or reducing the duodenal amplitude, wherein the method comprises administering pasteurized Akkermansia to a subject in need thereof, preferably in a therapeutically efficient dose without causing treatment limiting side effects.

Another object of the invention is a method for controlling, modulating, treating, or reducing the duodenal amplitude, wherein the method comprises administering pasteurized Akkermansia to a subject in need thereof and wherein the duodenal frequency remains substantially unchanged, preferably in a therapeutically efficient dose without causing treatment limiting side effects.

Another object of the invention is a method for controlling, modulating, treating, or reducing the intestinal glucose absorption or the jejunal glucose absorption, wherein the method comprises administering pasteurized Akkermansia to a subject in need thereof, preferably in a therapeutically efficient dose without causing treatment limiting side effects.

In one embodiment, the composition, the pharmaceutical composition, the cosmetic composition, or the medicament further comprises additional probiotic strains or species, such as, for example, bacterial probiotic strains or species. In another embodiment, the further strains are pasteurized. These probiotics include bacteria, or fungal strains or species, preferably yeast strains or species. In one embodiment, said additional probiotic strains or species are selected from those naturally present in the gut of the subject, preferably in the human gut, more preferably in the gut of healthy human subjects.

Examples of bacterial probiotic strains or species that may be used in the present invention include, but are not limited to Lactobacillus, Lacticaseibacillus, Lactococcus, Bifidobacterium, Veillonella, Desemzia, Christensenella, Allobaculum, Coprococcus, Collinsella, Citrobacter, Turicibacter, Sutterella, Subdoligranulum, Streptococcus, Sporobacter, Sporacetigenium, Ruminococcus, Roseburia, Proteus, Propionobacterium, Leuconostoc, Weissella, Pediococcus, Streptococcus, Prevotella, Parabacteroides, Papillibacter, Oscillospira, Melissococcus, Dorea, Dialister, Clostridium, Cedecea, Catenibacterium, Butyrivibrio, Buttiauxella, Bulleidia, Bilophila, Bacteroides, Anaerovorax, Anaerostipes, Anaerofilum, Enterobacteriaceae, Fermicutes, Atopobium, Alistipes, Acinetobacter, Slackie, Shigella, Shewanella, Serratia, Mahella, Lachnospira, Klebsiella, Idiomarina, Fusobacterium, Faecalibacterium, Eubacterium, Enterococcus, Enterobacter, Eggerthella, Dysosmobacter, Anaerobutyricum or Anaerobacterium.

Preferred probiotic strains are Akkermansia glycaniphila, E. halii, Lactobacillus, Lacticaseibacillus, Lactococcus, Bifidobacterium, Christensenella, Clostridium, Anaerostipes, Faecalibacterium, Eubacterium, Enterococcus, Enterobacter, Eggerthella, Dysosmobacter, Anaerobutyricum or Anaerobacterium.

Examples of prokaryote strains or species that may be used in the present invention include, but are not limited to Archaea, Firmicutes, Verrucomicrobia, Christensenella, Bacteroidetes (such as, for example, Allistipes, Bacteroides ovatus, Bacteroides splachnicus, Bacteroides stercoris, Parabacteroides, Prevotella ruminicola, Porphyromondaceae, and related genus), Proteobacteria, Betaproteobacteria (such as, for example, Aquabacterium and Burkholderia), Gammaproteobacteria (such as, for example, Xanthomonadaceae), Actinobacteria (such as, for example, Actinomycetaceae and Atopobium), Fusobacteria, Methanobacteria, Spirochaetes, Fibrobacteres, Deferribacteres, Deinococcus, Thermus, Cyanobacteria, Methanobrevibacteria, Peptostreptococcus, Ruminococcus, Coprococcus, Subdolingranulum, Dorea, Bulleidia, Anaerofustis, Gemella, Roseburia, Dialister, Anaerotruncus, Staphylococcus, Micrococcus, Propionobacteria, Enterobacteriaceae, Faecalibacterium, Bacteroides, Parabacteroides, Prevotella, Eubacterium, Bacilli (such as, for example, Lactobacillus salivarius and related species, Aerococcus, Granulicatella, Streptococcus bovis and related genus and Streptococcus intermedius and related genus), Clostridium (such as, for example, Anaerobutyricum hallii, Eubacterium limosum, Anaerobutyricum oehngenii and related genus) and Butyrivibrio.

Examples of fungal probiotic strains or species, preferably yeast probiotic strains or species that may be used in the present invention include, but are not limited Ascomycetes, Zygomycetes and Deuteromycetes, preferably from the groups Aspergillus, Torulopsis, Zygosaccharomyces, Hansenula, Candida, Saccharomyces, Clavispora, Bretanomyces, Pichia, Amylomyces, Zygosaccharomyces, Endomyces, Hyphopichia, Zygosaccharomyces, Kluyveromyces, Mucor, Rhizopus, Yarrowia, Endomyces, Debaryomyces, and/or Penicillium.

In one embodiment of the invention, the only one microbial strain or species, preferably bacterial strain, or species, comprised in the composition, pharmaceutical composition, cosmetic composition, or medicament is Akkermansia muciniphila.

In one embodiment of the invention, the composition, pharmaceutical composition, cosmetic composition, or medicament consists of pasteurized Akkermansia muciniphila.

In one embodiment of the invention, the composition, the pharmaceutical composition, the cosmetic composition, or the medicament further comprises a prebiotic.

Examples of prebiotics that may be used in the present invention include, but are not limited to polyphenols, inulin and inulin-type fructans, oligofructose, beta-glucans, xylose, arabinose, arabinoxylan, ribose, galactose, rhamnose, cellobiose, fructose, lactose, salicin, sucrose, glucose, esculin, tween 80, trehalose, maltose, mannose, mellibiose, mucus or mucins, raffinose, fructooligosaccharides, galacto-oligosaccharides, polyphenols, amino acids, alcohols, fermentable carbohydrates and any combinations thereof.

Other non-limiting examples of prebiotics include water-soluble cellulose derivatives, water-insoluble cellulose derivatives, unprocessed oatmeal, metamucil, bran, and any combinations thereof.

Examples of water-soluble cellulose derivatives include, but are not limited to, methylcellulose, methyl ethyl cellulose, hydroxyethyl cellulose, ethyl hydroxyethyl cellulose, cationic hydroxyethyl cellulose, hydroxypropyl cellulose, hydroxyethyl methylcellulose, hydroxypropyl methylcellulose, and carboxymethyl cellulose.

The composition of the invention may be administered by oral administration, rectal administration, administration via esophagogastroduodenoscopy, administration via colonoscopy, administration using a nasogastric or orogastric tube.

The composition may be administered orally as tablets, pills, capsules, soft gelatin capsules, sugarcoated pills, or dispersing tablets, effervescent tablets, or other solids.

The composition may be administered orally as a liquid, a drinkable solution, or a liposome.

In one embodiment, the composition of the invention further comprises excipients, diluent and/or carriers selected regarding the intended route of administration. Examples of excipients, diluent and/or carriers include, but are not limited to, water, phosphate buffer saline, anaerobic phosphate buffer saline, sodium bicarbonate, juice, milk, yogurt, infant formula, dairy product, coloring agents, such as, for example, titane dioxide (E171), iron dioxide (E172) and brilliant black BN (E151); flavoring agents; thickeners, such as, for example, glycerol monostearate; sweeteners; coating agents, such as, for example, refined colza oil, soya oil, peanut oil, soya lecithin or fish gelatin; diluting agents, such as, for example, lactose, monohydrated lactose or starch; binding agents, such as, for example, povidone, pregelatinized starch, gums, saccharose, polyethylene glycol (PEG) 4000 or PEG 6000; disintegrating agents, such as, for example, microcrystalline cellulose or sodium carboxymethyl starch, such as, for example, sodium carboxymethyl starch type A; lubricant agents, such as, for example, magnesium stearate; flow agent, such as, for example, colloidal anhydrous silica, etc.

In one embodiment of the invention, the composition of the invention is a pharmaceutical composition.

In another embodiment, the composition of the invention is a food additive, drink additive, dietary supplement, nutritional product, medical food, or nutraceutical composition.

### SHORT DESCRIPTION OF THE DRAWINGS

FIGS. 1 A and 1 B illustrate the duodenal contraction after an oral administration of a control vehicle or pasteurized Akkermansia (pAkk) during 12 weeks on (FIG. 1 A) ex vivo measurement of colon mechanical contraction frequency and (FIG. 1 B) ex vivo measurement of colon mechanical contraction amplitude, **p<0.01 vs HFD Vehicle. The associated p values were obtained using the t-test.

FIG. 2 shows the intestinal glucose absorption after oral administration of a control vehicle or pAkk during 12 weeks on ex vivo glucose absorption in jejunal everted sacs, *p≤0.05 vs HFD Vehicle. The associated p values were obtained using a t-test.

FIGS. 3 A and 3 B show the blood glucose homeostasis after an oral administration of a control vehicle or pAkk in 6 h-fasted mice on glycemia (FIG. 3 A) and HOMA index calculated as insulinemia (mU/L)×glycemia (mmol/L)/22.5 (FIG. 3 B) at week 11 of treatment, *p≤0.05 vs HFD Vehicle. p values were obtained using a t-test.

### DETAILED DESCRIPTION OF THE DRAWING

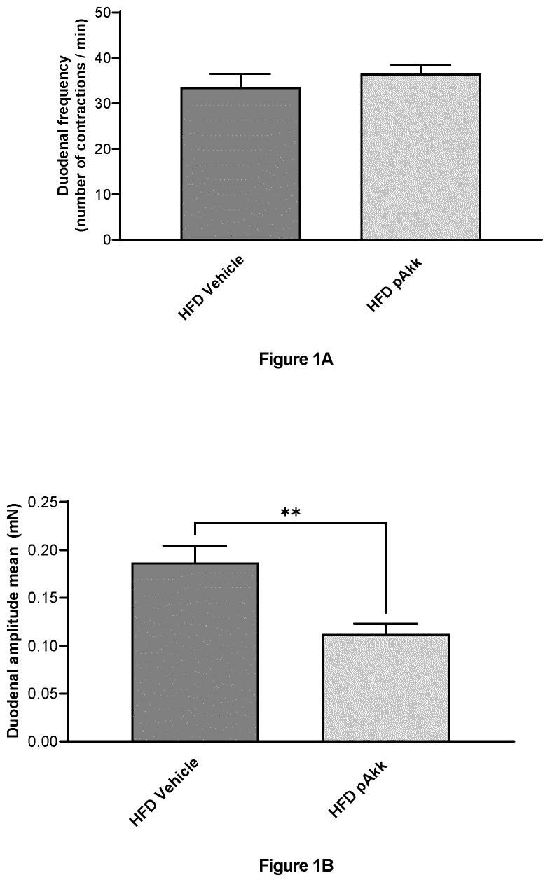

FIGS. 1 A and B, 2, and 3A and B, show the in vivo effect of administration of pasteurized Akkermansia muciniphila (in the Figures referred to as “pAkk”) on duodenum contraction, glucose absorption at the jejunum level in mice in a High Fat Diet-Fed mice, hereinafter referred to as “HFD”.

Nine-week-old male C57BL/6J mice (Charles River Laboratory, l'Arbresle, France) were allowed to at least 5 days of acclimatization period after the arrival. The animals were housed in ventilated and enriched cages (48×37.5×21 cm³) throughout the experimental phase. Animals' cages litters were changed once weekly. Mice were housed in groups of 5 animals on a normal light cycle (at 07:00 pm lights off), 22±2° C. and 50±10% relative humidity. Housing parameters were daily recorded. During the acclimation phase, standard diet (RM1 (E) 801492, SDS) and tap water were provided ad libitum. During the experimental phase (12 weeks), High fat diet 45% (Research Diet #12451) (HFD group) and tap water were provided ad libitum. Mice were treated daily with an oral gavage of 180 μL of vehicle, or 180 μL of pasteurized A. muciniphila solution (1.18*109 bacterial cells/day/mice) for the 12 weeks of HFD treatment.

Duodenal contraction: At the end of the treatment, duodenum segments were dissected, washed, and incubated in oxygenated Krebs-Ringer solution for 30 min at 37° C., attached to the isotonic transducer (MLT7006 Isotonic Transducer, Hugo Basile, Comerio, Italy), and immersed in an organ bath of the same medium maintained at 37° C. The load applied to the lever was 1 g (10 mN). Isotonic contractions were recorded on Labchart software (AD Instruments) following the transducer displacement. After attaching the intestinal segments, contractions were recorded for 15 min. The basal contractions were presented as average of amplitude and frequency of contraction.

Duodenum and Jejunum glucose absorption: At the end of two-hour fasted the duodenum and the jejunum were then harvested, washed, everted, and filled with a Krebs-Ringer solution without glucose. Everted duodenal sacs were incubated in Krebs-Ringer with 10 g/L of glucose for 2 min at 37° C. The media of each sac was then collected and immediately frozen for subsequent glucose quantification studies. Glucose was measured using Glucose GOD FS 10′kit (DiaSys, France).

Diabetes is generally associated with an intestinal hyper-contractility that favors hyperglycemia and insulin-resistance.

Surprisingly, in HFD mice, the daily oral administration of pAkk have the capacity to decrease significantly duodenal amplitude hypercontractility (FIG. 1) compared to HFD Vehicle.

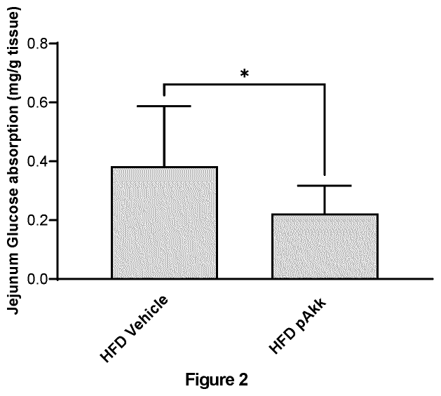

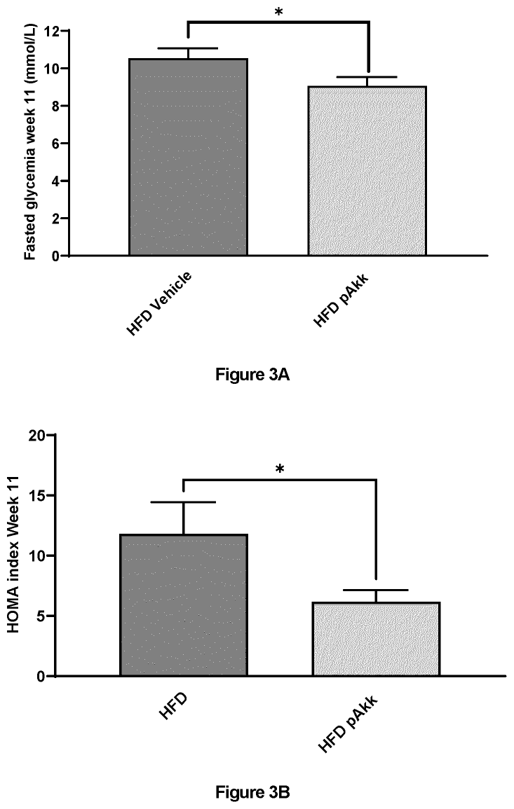

However, no significant effect was observed on the duodenal contraction frequency. This impact of duodenal amplitude contraction is both associated with a significant decrease of jejunal glucose absorption (FIG. 2) and decrease of fasted blood glucose and insulin-resistance (HOMA index) (FIG. 3) in response to pAkk compared to HFD vehicle.

### Translation of the English Expressions in the Drawings:

| | |
|---|---|
| English expression | Translation |

Duodenal frequency
number of contractions/min
HFD vehicle
HFD pAkk
Duodenal amplitude mean (mN)
Jejunum Glucose absorption mg/g tissue
Fasted glycemia week 11 (mmol/L)
HOMA index Week 11

1. Composition comprising pasteurized Akkermansia for use in the prevention or treatment of gut contractility disorders.

2. Composition for use in the prevention or treatment of gut contractility disorders according to claim 1, wherein the pasteurized Akkermansia is Akkermansia muciniphila.

3. Composition for use in the prevention or treatment of gut contractility disorders according to claim 1, wherein the duodenal amplitude is controlled, modulated, or reduced in patients suffering from gut contractility disorders.

4. Composition for use in the prevention or treatment of gut contractility disorders according to claim 1, wherein the jejunum glucose absorption is controlled, modulated, or reduced in patients suffering from gut contractility disorders.

5. Composition for use in the prevention or treatment of gut contractility disorders according to claim 1, wherein the jejunum glucose absorption is controlled or reduced in diabetes or obesity patients suffering from gut contractility disorders.

6. Composition for use in the prevention or treatment of gut contractility disorders according to claim 1, wherein pasteurized Akkermansia is administered for 2 to 10 days, preferably for 3 to 7 days and even more preferably for 4 to 6 days.

7. Composition for use in the prevention or treatment of gut contractility disorders according to claim 1, wherein the pasteurized Akkermansia is administered in an amount from 1·10⁴ to 1·10¹² cells per day, more preferably from 1·10⁵ cells to 1·10¹¹ cells per day, and even more preferably from 1·10⁶ to 5·10¹⁰ cells per day.

8. Composition for use in the prevention or treatment of gut contractility disorders according to claim 1, wherein the pasteurized Akkermansia is administered in an amount from 1·10⁸ to 5·10¹⁰ cells per day.

9. Composition for use in the prevention or treatment of gut contractility disorders according to claim 1, further comprising one or more ingredients chosen from group consisting of probiotic, bacteria, yeast, microorganisms, prebiotic, or a combination thereof.

10. Composition for use in the prevention or treatment of gut contractility disorders according to claim 1, wherein the composition further comprising a mineral or a vitamin or a combination thereof.

11. Composition for use in the prevention or treatment of gut contractility disorders according to claim 1, wherein the composition further comprises a pharmaceutically acceptable carrier or a food grade carrier.

12. Composition for use in the prevention or treatment of gut contractility disorders according to claim 1, wherein the composition is orally administered.

13. Composition for use in the prevention or treatment of gut contractility disorders according to claim 1, wherein the composition is a cosmetic composition, a nutritional composition, a food product, a dietary complement, a medical food, or a medicament.

14. Composition for use in the prevention or treatment of gut contractility disorders according to claim 1, wherein the composition further comprises a plant extract, chosen from the group consisting of Camellia sinensis, Aronia melanocarpa, Emblica officinalis, Olea europa, Citrus bergamia, Vaccinium macrocarpon, Myrciaria dubia, red Panax ginseng, Vaccinium oxycoccos, Vaccinium macrocarpon.

15. Non-medical use of the composition according to claim 1, or reducing gut contractility disorders.
